(19) United States
(12) Patent Application Publication
(10) Pub. No.: US 2005/0032728 A1
(43) Pub. Date: Feb. 10, 2005

(54) Tumor suppression through bicistronic co-expression of p53 and p14ARF

(75) Inventors: Ruth A, Gjerset, San Diego, CA (US); Yinghui Huang, San Diego, CA (US); Neshat Saadatmandi, San Diego, CA (US)

Correspondence Address: McDermott Will & Emery LLP., 4370 La Jolla Village Drive, Suite 700, San Jose, CA, 92122, US

(73) Assignee: Sidney Kimmel Cancer Center

(21) Appl. No.: 10/717,845

(22) Filed: Nov. 19, 2003

Related U.S. Application Data
(60) Provisional application No. 60/434,267, filed on Dec. 17, 2002.

(51) Int. Cl. A61K 48/00; A61K 9/127; C12N 15/86

(52) U.S. Cl. 514/44; 424/450; 435/320.1; 435/456

(57) ABSTRACT

A bicistronic construct of p53 and p14ARF, vectors and other delivery vehicles which include the bicistronic construct, and methods of treating cancer using the vectors and bicistronic construct.

### BACKGROUND OF THE INVENTION

This application is based on provisional U.S. application No. 60/434,267, filed Dec. 17, 2002.

1. Field of the Invention

The present invention relates generally to the field of cancer therapy.

2. Description of Related Art

In the past, p53 gene transfer to tumors (p53 gene therapy) has been attempted by incorporating the gene for p53, usually coupled to an appropriate transcriptional promoter DNA sequence, into a viral or non-viral vectors. The vector promotes the entry of the p53 gene into the cancer cell, where it is transcribed and translated into p53 protein. A preferred embodiment of p53 gene therapy has used replication-impaired adenoviral vectors derived from Type V human adenovirus, in which the early region E1A/B genes of the viral genome required for viral replication are replaced by the wild-type p53 gene and appropriate promoter sequence. Adenoviral vectors have advantages for industrial production, as they are relatively stable and easy to prepare in high titer. Adenoviruses are able to enter most cell types, and therefore can be used as delivery vehicles for DNA. These vectors have shown efficacy against tumors in several animal models and have been used in clinical trials in humans for various cancers. Saadatmandi N, Wilson D R, Gjerset R A. p53 Gene Therapy. Encyclopedia of Cancer: Academic Press, 2002;425-432.

Often, treatment with the Adenoviral p53 vectors fails to achieve complete eradication of the tumor and must be used in combination with a conventional DNA damaging chemotherapy to enhance p53 activity. There are reports that the combination approach in patients with tumors that had previously failed conventional therapy can result in improved responses compared with single agent treatment (i.e., either conventional therapy alone, or p53 adenovirus alone) Saadatmandi, N., Wilson, D. R., and Gjerset, R. A. p53 Gene Therapy. In: J. R. Bertino (ed.) Encyclopedia of Cancer, second edition, Vol. 3. San Diego: Academic Press, 2002. Nevertheless, the p53 plus chemotherapy combined approach again requires that patients receive conventional chemotherapy, and be exposed to the toxic side effects encountered with this form of therapy.

The p14ARF tumor suppressor is known to act at least in part by stabilizing p53 and increasing its activity (Zhang, Y., Xiong, Y., and Yarbrough, W. G. ARF promotes MDM2 degradation and stabilizes p53: ARF-INK4a locus deletion impairs both the Rb and p53 tumor suppression pathways, Cell. 92: 725-34., 1998; Kamijo, T., Weber, J. D., Zambetti, G., Zindy, F., Roussel, M. F., and Sherr, C. J. Functional and physical interactions of the ARF tumor suppressor with p53 and Mdm2, Proc Natl Acad Sci USA. 95: 8292-7., 1998.) In one study, p14ARF was found to be suppressive of tumor cells that retained expression of wild-type p53 and lost expression of p14ARF and were refractory to p53 gene transfer (Lu, W., Lin, J., and Chen, J. Expression of p14ARF overcomes tumor resistance to p53, Cancer Res. 62: 1305-10., 2002) In another study, co-expression of p53 and p14ARF, delivered separately in independent vectors was better than either vector alone in tumors cells that had lost either wild-type p53 or p14ARF expression (Tango, Y., Fujiwara, T., Itoshima, T., Takata, Y., Katsuda, K., Uno, F., Ohtani, S., Tani, T., Roth, J. A., and Tanaka, N. Adenovirus-Mediated p14ARF Gene Transfer Cooperates with Ad5CMV-p53 to Induce Apoptosis in Human Cancer Cells, Hum Gene Ther. 13: 1373-82., 2002)

Nearly all cancer cells survive by losing the p53 pathway, either through loss or mutation of the p53 gene or through deregulation of the pathway in another way. Restoration of the pathway through p53 gene transfer is generally suppressive of cancer cells. Gjerset R A, Turla S T, Sobol R E, et al. Use of wild-type p53 to achieve complete treatment sensitization of tumor cells expressing endogenous mutant p53. Mol Carcinog 1995;14:275-85; Gjerset R A, Mercola D. Sensitization of tumors to chemotherapy through gene therapy. Adv Exp Med Biol 2000;465 :273-91. The pathway is latent in normal cells because the signals that trigger its activation, including activation of oncogenes, are absent, and gene transfer of p53 is much less suppressive of normal cells (Katayose, et al., Cytotoxic effects of adenovirus-mediated p53 protein expression in normal and tumor mammary epithelial cells., Clin. Cancer Res. 1(8): 889-897, 1995.). P53-based therapies, including p53 gene therapy have therefore attracted interest as a potentially highly efficacious tumor-specific therapy with reduced toxicity.

Conventional treatments are presently unable to achieve cures for most cancers. Cancer, Principles and Practice of Oncology. DeVita, V. T., Hellman, S., Rosenberg, S. A., eds., J.B. Lippincott Comp., Philadelphia. Sixth edition (2001). Furthermore, because these treatments often target cellular pathways shared by normal cells, they can be extremely toxic to normal tissue. A potentially more effective approach to cancer treatment would target cellular processes, such as the p53 pathway, to which cancer cells might be uniquely or preferentially susceptible. A single gene p53 replacement strategy to tumor suppression is often ineffective (Gjerset, R. A., Turla, S. T., Sobol, R. E., Scalise, J. J., Mercola, D., Collins, H., Hopkins, P. Use of wild-type p53 to achieve complete treatment sensitization of tumor cells expressing endogenous mutant p53, Molecular Carcinogenesis, 14:275-285, 1995.) and a p53 plus chemotherapy combination approach again requires that patients receive conventional chemotherapy, and be exposed to the toxic side effects encountered with this form of therapy. There is therefore a need for new therapeutic approaches to cancer the exploit tumor suppressor genes or suppressor gene combinations more effectively and that have reduced toxicity as well as increased efficacy, compared to conventional treatments.

### SUMMARY OF THE INVENTION

The invention is directed to a method of inducing killing, or apoptosis, or growth arrest of malignant or metastatic cancer cells. The method involves contacting cancer cells with a bicistronic construct of p53 and p14ARF genes (or gene variants thereof), which express protein having tumor suppressor activity. The method may be used in combination with one or modes of therapy, such as radiation therapy and chemotherapy.

Another aspect of the invention is a bicistronic construct comprising p53 and p14ARF genes or gene variants thereof. An embodiment includes the bicistronic construct disposed in a viral vector selected from the group of vectors consisting of retro viral, adeno-associated viral, herpes simplex viral, cytomegaloviral vectors. The bicistronic construct may be disposed in a non-viral delivery vehicle selected from the group consisting of liposomes, polylysine carrier complexes, or naked DNA. Viral vectors and non-viral delivery vehicles which comprise the bicistronic construct are subjects of the invention. Another composition of the invention includes a pharmaceutical carrier which contains either a bicistronic construct comprising p53 and p14ARF genes, or a vector comprising a bicistronic construct comprising p53 and p14ARF genes, or a non-viral delivery vehicle comprising a bicistronic construct comprising p53 and p14ARF genes.

### BRIEF DESCRIPTION OF THE DRAWINGS

FIG. 1 is a diagram of the method of the invention in related to other approaches in the art.

FIG. 2 is a diagram showing construction of p53/p14ARF bicistronic adenoviral vector.

FIG. 3. Percent viability measured in 96-well viability assays using MTS, at 72 hours following treatment with the indicated doses of AdLuc, Adp53, or Adp14/p53 for (A) DLD-1 cells, and (B) N₂O₂ cells. Each data point represents the average of triplicate samples, with standard deviations shown (for some points standard deviations are less than the size of symbol). Data is normalized to viability measured in control, untreated wells. (C) Similar assay carried out on DLD-1 cells treated singly with either Adp53 or Adp53 at the indicated doses, or with a combination of the two vectors, each at the indicated dose.

FIG. 4. (A) Trypan Blue exclusion assay of DLD-1 cells and N₂O₂ cells 48 and 72 hours after treatment with AdLuc. Adp53, or Adp14/p53 at 20 pfu/cell, or Adp53 at 200 pfu/cell. Date points represent the average of duplicate wells. (B) FACS analysis of propidium iodide stained cells harvested 48 hours after treatment with 20 pfu/cell of AdLuc, Adp53, or Adp14/p53.

FIG. 5. Growth of subcutaneous tumors of N₂O₂ cells in nude mice following treatment with AdLuc (control vector) or Adp14/p53 (bicistronic vector). Arrows indicate days of intratumoral administration of vector. Tumors were established by subcutaneous implantation of 10⁶ tumor cells and allowed to grow to a size of about 30 mm³ before treatment was initiated.

### DETAILED DESCRIPTION OF THE INVENTION

Our approach was to focus on a cellular pathway regulated by the p53 tumor suppressor that leads to cell death or cell growth arrest in response to certain cellular abnormalities commonly encountered in cancer cells, such as DNA damage and oncogene expression.

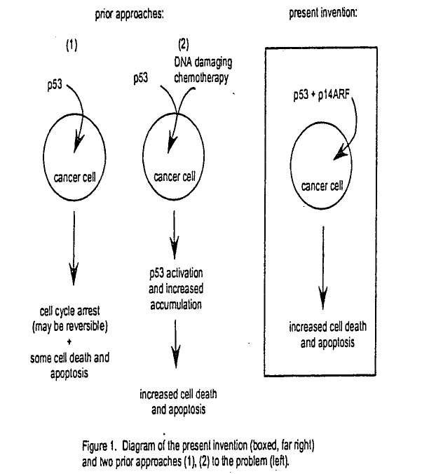

We found that a bicistronic construct of p53/p14ARF was superior to either of two single gene vectors (for either p53 or p14ARF, respectively) to enhance p53 activity, and was surprisingly better than a combination of two single gene vectors for p53 and p14ARF. In cells expressing endogenous p14ARF, or expressing both endogenous ARF and wild-type p53, there was a striking improvement in tumor suppression by supplying exogenous p14ARF together with p53 as a bicistronic construct expressing the two proteins. The method of the invention, in comparison with other approaches in the art, is illustrated in FIG. 1.

The vector is structurally different than prior vectors in that it incorporates both the p53 and p14ARF genes in a single expression cassette under the control of a single promoter. In our invention, the two genes are co-delivered as one vector, rather than as single gene vectors. Functionally, the bicistronic vector is superior either to p53 alone, or to a combination of single gene vectors for p53 and p14ARF.

Our vector would be used to deliver the p53 and p14ARF genes to human malignant or metastatic cancer cells so as to induce killing or apoptosis or growth arrest in these cells. While a preferred embodiment of the present invention involves delivery of a p14ARF and p53 to the tumor via an adenoviral vector, we also anticipate that other delivery mechanisms, including retro viral, adeno-associated viral, herpes simplex viral, cytomegaloviral, could also be used. Methods for formulating pharmaceutical compositions or carriers for the bicistronic constructs or vectors disclosed herein are well known in the art (e.g. U.S. Pat. Nos. 6,054,467, and 5,747,469) incorporated herein by reference). Non-viral delivery vehicles could be used as well, including approaches that utilize liposomes, polylysine carrier complexes, or naked DNA (1992, Proc. Natl. Acad. Sci. USA 89:6099-6103; Zhu et al., Systemic gene expressino after intravenous DNA delivery into adult mice, (1993) Science 261:209-211; Yoshimura et al., (1992) Nucleic Acids Research 20: 3233-3240). Methods for combination therapy involving chemotherapy and gene therapy are well known (e.g. U.S. Pat. Nos. 6,054,467; 5,747,469)

We have used the cytomegalovirus promoter to achieve expression of p53 and p14ARF, but other promoters could be used as well, including the Rous Sarcoma Virus promoter, and SV40 promoter. In some embodiments, the vector could be used in combination with radiation, and/or with conventional chemotherapy of all types such as cisplatin, etoposide, camptothecin, doxorubicin, 5-fluorouracil. Our invention would also include variants of p53 or p 14ARF (such as mutated or truncated forms of these tumor suppressors) that retain the tumor suppressor activity of the protein, or that display enhanced tumor suppressor activity.

We anticipate that all types of human tumors, irrespective of their endogenous p53 and ARF status, would be amenable to this approach. Preferred embodiments would be head and neck cancer, breast cancer, and lung cancer.

### EXAMPLE

We constructed a bicistronic adenovirus encoding ARF and p53 (denoted Adp14/p53 or AdBi-cis), using the AdEasy kit of Quantum Biotechnologies. The p14ARF and p53 coding sequences were obtained from normal human fibroblast RNA by RT-PCR amplification. The bicistronic cassette containing an internal ribosome entry site (IRES) flanked by multicloning sites was obtained from the pIRES vector of Clontech.

To obtain the full length adenoviral genome, the pSHUTTLE-CMV construct containing the ARF-IRES-p53 insert was recombined in bacteria with the AdEasy vector (which encodes the remainder of the adenoviral genome minus E1 and E3, followed by packaging in 293 kidney cells.

We constructed the bicistronic vector for p14ARF and p53 as described above (see FIG. 2). In a similar manner we prepared adenoviral vectors encoding full length ARF alone (Adp14). An adenovirus encoding the p53 tumor suppressor under the control of the Cytomegalovirus promoter was provided by Introgen Therapeutics, Inc.

The vectors were expanded, purified and titered. We demonstrated that each vector was able to induce expression of its respective transgene following treatment of tumor cells. We then carried out viability assays with several tumor cell lines to determine the relative tumor suppressor activity of the various vectors. Tumor cells growing as monolayer cultures in vitro were exposed for 4 hours to various doses of the bicistronic adenoviral vector Adp14/p53 described above, replated at low density in 96 well plates (triplicate wells for each vector treatment) and viability was scored 3 days later by a standard MTS assay, which measures the bioconversion of a formazan compound to a colored derivative that absorbs at 490 nm. Absorbance is proportional to the number of viable cells. We express viability as a percentage of the viability of untreated cells.

The cell lines tested were DLD-1 human colon cancer cells, that express endogenous mutant p53 and endogenous ARF, and murine N202 cells that express endogenous wild-type p53 and endogenous ARF.

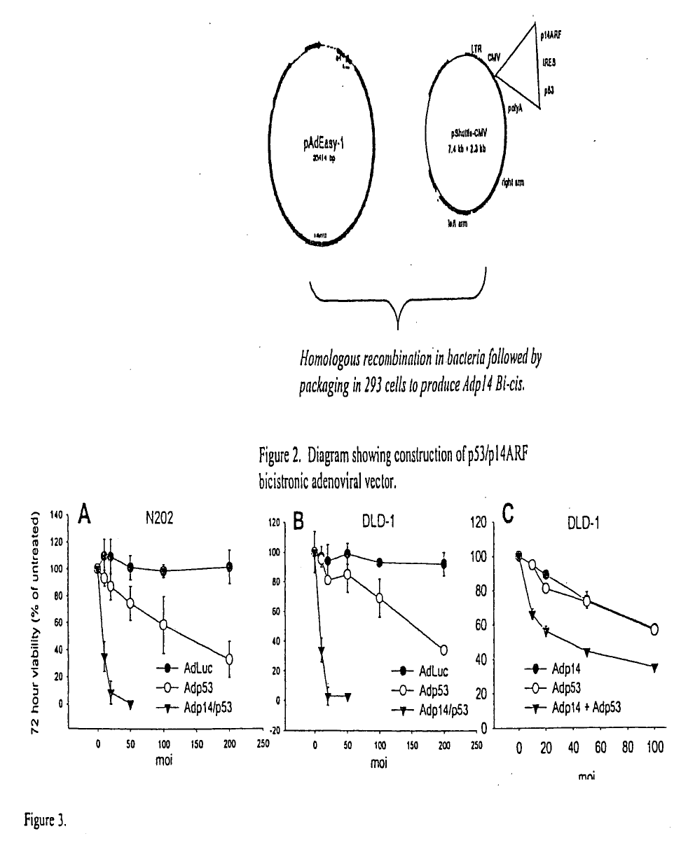

We found that the growth and viability of tumor cells was completely suppressed by treatment with a bicistronic adenoviral vector encoding p53 and p14^{ARF} (denoted “ARF”) and that the doses of vector needed to achieve complete suppression were 20 times lower than doses needed to achieve suppression with single gene vectors for p53 or ARF (FIG. 3A,B). Unexpectedly, we found that a bicistronic vector was more effective than a combination of the two single gene vectors, when each was provided at a dose equivalent to the dose of bicistronic vector (FIG. 3C). The activity of the bicistronic ARF/p53 vector did not appear to depend on the endogenous p53 or ARF status of the tumor cell, as the cell lines shown in FIG. 3 expressed either wild-type p53 (N₂O₂) or mutant p53 (DLD-1) and both cell lines expressed endogenous p14ARF. The advantages of such a vector are that (1) it enabled us to exploit the combined potential of p53 and ARF, without increasing vector doses, (2) it achieved greater anti-tumor efficacy with less vector, thus reducing the possibility of adverse vector-related side effects, and (3) it is likely to have a very broad application to tumor cells of varying p53 and ARF status. There are presently no reports of anti-tumor vectors of this type.

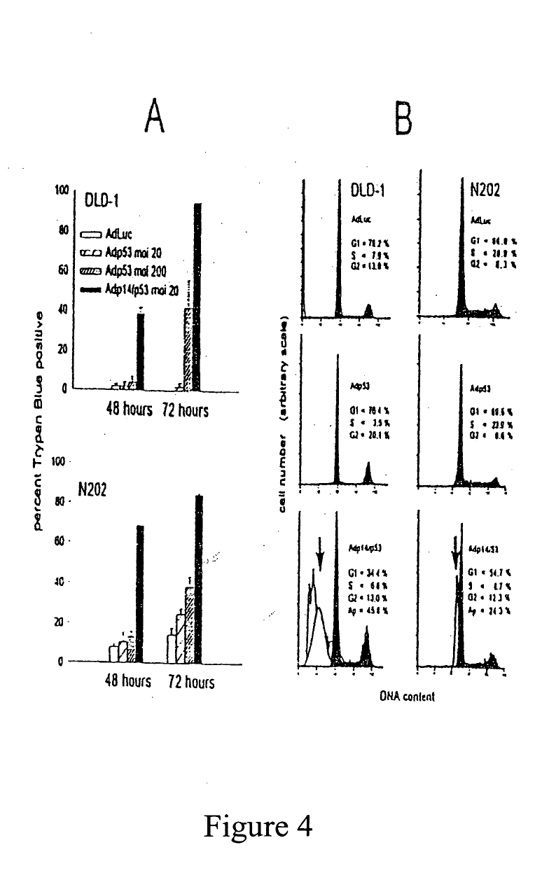

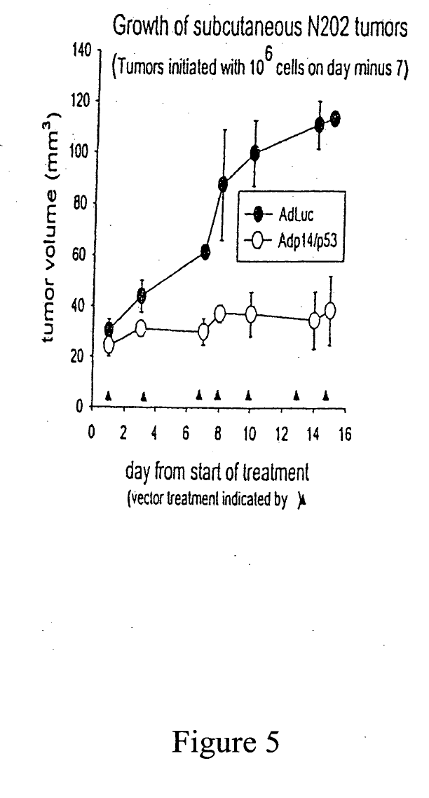

Also surprising was our ability to completely abolish the viabilty of several tumor cell lines of different origins in vitro using very low vector to cell ratios (less than 20). Suppression of growth and viability was associated with the induction of cell death, as evidenced by a dye exclusion assay (Trypan Blue), where dead cells appear Trypan Blue positive (FIG. 4A), and by Flow cytometry analysis of DNA contact, where cells undergoing cell death through apoptosis appeared as a peak with reduced DNA content (FIG. 4B, arrows). We also found that intratumoral administration of the bicistronic vector to established subcutaneous tumors in nude mice derived from murine N202 breast cancer cells, resulted in a dramatic suppression of tumor growth (FIG. 5).

In the experiment shown in FIG. 5, we implanted 10⁶ tumor cells subcutaneously in nude mice and waited until tumors had reached a size of about 30 mm³ based on length and width measurements of tumors and estimated using the formula volume=12 (length)×(width)². We then injected 10⁸ pfu (plaque forming units) of vector per tumor by intratumoral injection. We repeated the treatments at two to three day intervals. Control animals received a similar treatment of a similar adenoviral vector expressing firefly luciferase. Tumors treated with the control vector continued to grow over the course of the treatment, whereas tumors treated with the bicistronic vector barely increased in size. This result was surprising in light of our earlier studies with a p53 adenovirus, where treatment doses in the range of 10⁸ pfu/cell failed to achieve significant reduction in subcutaneous tumor growth of several tumor types unless DNA damaging chemotherapy was included in the treatment regimen. (Gjerset, R. A. and Mercola, D. Sensitization of tumors to chemotherapy through gene therapy, Adv Exp Med Biol. 465: 273-91, 2000; Lebedeva, S., Bagdasarova, S., Tyler, T., Mu, X., Wilson, D. R., and Gjerset, R. A. Tumor suppression and therapy sensitization of localized and metastatic breast cancer by adenovirus p53, Hum Gene Ther. 12: 763-72., 2001).

By ensuring that the p53 pathway was maximally induced, the bicistronic vector provided a highly improved biological approach to cancer therapy, compared to single gene treatments, and was far better even than a combination of single gene treatments. The high degree of anti-tumor efficacy achieved with the bicistronic vector may obviate the need to combine this highly targeted biological treatment with conventional chemotherapy or radiation, as has been necessary in the past to optimize the single gene approach for p53 (see overview article Saadatmandi, N., Wilson, D. R., and Gjerset, R. A. p53 Gene Therapy. In: J. R. Bertino (ed.) Encyclopedia of Cancer, second edition, Vol. 3. San Diego: Academic Press, 2002). In some cases, however, a combination with conventional treatments may further enhance the anti tumor benefits of this therapy. The bicistronic vector or an alternative delivery vector for a bicistronic expression cassette encoding p53 and p14ARF is anticipated to be broadly applicable to the treatment of a wide range of cancers.

The references cited herein, to the extent that they provide exemplary procedural or other details supplementary to those set forth herein, are specifically incorporated by reference.

1. A bicistronic construct comprising p53 and p14ARF genes or gene variants thereof.

2. The bicistronic construct of claim 1 disposed in a vector selected from the group of vectors consisting of retro viral, adeno-associated viral, herpes simplex viral, cytomegaloviral vectors.

3. The bicistronic construct of claim 1 disposed in a non-viral delivery vehicle selected from the group consisting of liposomes, polylysine carrier complexes, or naked DNA.

4. A vector comprising a bicistronic construct comprising p53 and p14ARF genes, or genes variants thereof, wherein said vector is selected from the group of vectors consisting of retro viral, adeno-associated viral, herpes simplex viral, cytomegaloviral vectors.

5. A delivery vehicle comprising a bicistronic construct comprising p53 and p14ARF genes, or genes variants thereof, wherein said delivery vehicle is selected from the group consisting of liposomes, polylysine carrier complexes, or naked DNA.

6. A pharmaceutical carrier comprising a bicistronic construct comprising p53 and p14ARF genes or genes variants thereof, or a vector comprising a bicistronic construct comprising p53 and p14ARF genes or genes variants thereof, or delivery vehicle comprising a bicistronic construct comprising p53 and p14ARF genes or genes variants thereof, wherein said delivery vehicle is selected from the group consisting of liposomes, polylysine carrier complexes, or naked DNA.

7. A method of inducing killing or apoptosis or growth arrest of malignant or metastatic cancer cells, said method comprising the step of contacting said cells with a bicistronic construct comprising p53 and p14ARF genes, or gene variants thereof provided that said variants express protein having tumor suppressor activity.

8. The method of claim 7 wherein said bicistronic construct is disposed in a vector selected from the group of vectors consisting of retro viral, adeno-associated viral, herpes simplex viral, cytomegaloviral vectors.

9. The method of claim 7 wherein said bicistronic construct is disposed in a non-viral delivery vehicle selected from the group consisting of liposomes, polylysine carrier complexes, or naked DNA.

10. The method of claim 7 wherein said bicistronic construct is disposed in a pharmaceutical carrier.

11. The method of claim 10 wherein said bicistronic construct is disposed in a viral vector selected from the group of vectors consisting of retro viral, adeno-associated viral, herpes simplex viral, cytomegaloviral vectors.

12. The method of claim 10 said bicistronic construct is disposed in a non-viral delivery vehicle selected from the group consisting of liposomes, polylysine carrier complexes, or naked DNA.

13. The method of claim 7 used in combination with one or modes of therapy selected from the group consisting of radiation therapy and chemotherapy.

14. The method of claim 7 wherein said cancer cell is selected from the group consisting of head and neck cancer cells, breast cancer cells, lung cancer cells, colon tumor cells, liver tumor cells, brain tumor cells, kidney tumor cells, skim tumor cells, ovarian tumor cells, prostate tumor cells.
